(19) United States
(12) Patent Application Publication
(10) Pub. No.: US 2010/0233727 A1
(43) Pub. Date: Sep. 16, 2010

(54) MARKER FOR ARRHYTHMIA RISK

(76) Inventors: Samuel C. DUDLEY, JR., Chicago, IL (US); Heather L. BLOOM, Atlanta, GA (US)

Correspondence Address: BLANK ROME LLP, WATERGATE, 600 NEW HAMPSHIRE AVENUE, N.W., WASHINGTON, DC, 20037, US

(21) Appl. No.: 12/725,590

(22) Filed: Mar. 17, 2010

Related U.S. Application Data
(63) Continuation-in-part of application No. 12/207,985, filed on Sep. 10, 2008.
(60) Provisional application No. 60/960,013, filed on Sep. 11, 2007.

(51) Int. Cl. G01N 33/53; C12Q 1/02

(52) U.S. Cl. 435/7.1; 435/29

(57) ABSTRACT

The present invention relates to markers and methods for determining risk of ventricular arrhythmia in an African American or woman patient. By using the markers of the present invention, individual with high risk of ventricular arrhythmia can properly be detected and treated. The present inventors have discovered that, in African American and women, IL-6 and/or DROMs and/or CRP have strongly positive correlation with the risk of ventricular arrhythmia.

This application is a continuation-in-part (CIP) of co-pending U.S. patent application Ser. No. 12/207,985, filed Sep. 10, 2008, which claims the benefit of U.S. Provisional Patent Application No. 60/960,013, filed Sep. 11, 2007, the disclosures of which are incorporated herein by reference.

### FIELD OF THE INVENTION

The present invention relates to markers and methods for determining risk of ventricular arrhythmia in an individual. By using the markers of the present invention, individual with high risk of ventricular arrhythmia can properly be detected and treated.

### BACKGROUND OF THE INVENTION

Sudden Cardiac death (SCD) accounts for more than 50% of cardiac-related death¹, numbering over 400,000 deaths per year² in the United States. Ventricular arrhythmias cause most of these deaths³. The only treatment for ventricular arrhythmias with proven mortality benefit is the internal cardioverter-defibrillator (ICD). Two recent observational trials have demonstrated that Hydroxymethylglutaryl coenzyme A reductase inhibitors (statins) decrease the incidence of ventricular arrhythmias and increase survival in patients with ICDs^{4;5}. This survival benefit exists for both ischemic (MADITII) and non-ischemic cardiomyopathy (DEFINITE). The reduction in ICD discharges is independent of the cholesterol-lowering effects.

One proposed mechanism for the anti-arrhythmic effect of statins is their anti-oxidant properties⁴. Statins reduce the generation of reactive oxygen species by inhibition of vascular NAD(P)H oxidase^{6;7}, inhibit the respiratory burst of phagocytes⁸, antagonize the pro-oxidant effect of angiotensin II and endothelin-1⁹, and increase the synthesis of vascular nitric oxide^{10;11}. In addition, some statins and their metabolites are direct free radical scavengers. Statins may also have important anti-inflammatory effects. As inflammation is closely linked to the production of reactive oxygen species (ROS), the molecular basis of the observed anti-inflammatory effects of statins may relate to their ability block the production and/or activity of ROS.¹²

Several lines of evidence link oxidative stress with arrhythmias. ¹³⁻¹⁵. H₂O₂, a form of oxidative stress, causes alterations in cellular electrophysiology resulting in increased ventricular arrhythmias. H₂O₂ reduces sodium channel current and prevents its complete inactivation, causing a persistent current during the action potential plateau. This effect appeared to be the result of lipid peroxidation¹⁶. Patch clamp experiments in rat myocytes have also observed a H₂O₂ induced augmentation of sodium current via a slowing of the inactivation kinetics, producing a marked prolongation of the cellular action potential¹⁷. This provides good reason to believe that statins act to reduce arrhythmic risk, in part, by reducing lipid peroxidation.

Treatment with statin and/or ICD, however, is not always necessary, currently, ventricular arrhythmic risk is determine by the ejection fraction. Generally, an ejection fraction (EF) lower than about 35% is a risk factor for ventricular arrhythmia; however, many patients with EF less than about 35% does not have ventricular arrythmia. Nevertheless, out of abundant of caution, these patients receive ICD and/or statin treatment. Therefore, there remains a need for an independent and simple test for diagnosing and assessing ventricular arrhythmic risk, possibly as a supplement to EF, to reduce the number of unnecessary treatment.

### SUMMARY OF THE INVENTION

An object of the present invention is to provide a method for assessing or diagnosing the risk of ventricular arrhythmia in a subject.

Another object of the present invention is to provide a method for preventing or substantially reducing the risk of ventricular arrhythmia in a subject.

The present invention relates to markers and methods for determining risk of ventricular arrhythmia in a subject, preferably a person. The present inventors have discovered that derivative of reactive oxidative metabolites (DROMs) and/or interleukin-6 (IL-6) are significant markers for ventricular arrhythmic risk. Thus, an abnormally high concentration of DROMs and/or IL-6 indicate a high risk of ventricular arrhythmic risk. “Abnormally high” is used herein to mean that the concentration is significantly higher than the average concentration in normal individuals without ventricular arrhythmia, preferably about 20% higher the normal concentration. For African Americans and women, in additional to DROMs and/or IL-6, C-reactive protein (CRP) is also a marker for ventricular arrhythmic risk. Here, an abnormally high concentration of CRP indicates and elevated risk of ventricular arrhythmic risk.

In accordance with the present invention, a sample, preferably a blood sample, is taken from a subject. The concentration of DROMs and/or IL-6 and/or CRP in the sample is measured and compared to concentrations in normal subjects. If the concentration is abnormally high, then the subject is assessed or diagnosed as having a high risk of ventricular arrhythmia.

The method of the present invention can be used alone or in conjunction with the commonly used ejection fraction (EF) to assess or diagnose ventricular arrhythmic risk. When used in conjunction with the EF test, patients at risk for ventricular arrhythmic risk would have abnormally high concentration of DROMs and/or IL-6 and/or CRP and an EF less than about 35%. The present methods are best suited to confirm assessment and diagnosis by EF measurement. Other methods used to diagnose arrhythmic risk known in the art can also be used with the markers of the present invention.

### BRIEF DESCRIPTION OF THE DRAWINGS

FIG. 1 is a graph comparing ICD event for statin and non-statin users.

FIG. 2 is a graph comparing EF, hsCRP, DROM and IL-6 by statin use.

FIG. 3 is a graph showing incidence of appropriate ICD shocks and antitachycardia pacing in women and men.

### DETAILED DESCRIPTION OF THE PREFERRED EMBODIMENT

To practice the present invention, the following steps are performed: 1) taking a sample, preferably a blood sample from a subject; 2) determine the concentration of reactive oxidative metabolites (DROMs) and/or interleukin-6 (IL-6) in the sample; and 3) diagnosing or assessing high ventricular arrhythmia risk when the DROMs and/or IL-6 concentration is abnormally high. For an African American or a women subject, the steps include 1) taking a sample, preferably a blood sample from a subject; 2) determine the concentration of reactive oxidative metabolites (DROMs) and/or interleukin-6 (IL-6) and/or C-reactive protein (CRP) in the sample; and 3) diagnosing or assessing high ventricular arrhythmia risk when the DROMs and/or IL-6 and/or CRP concentration is abnormally high. Although the markers can be used individually to diagnose or assess high ventricular arrhythmia risk, in an embodiment of the present invention, the markers can be used together to accurately diagnose of assess high ventricular arrhythmia risk.

DROMs concentration can be determined as disclosed by Cessarone et al (Int. Angio. 2:127-130, 1999), Alberti et al. (Res. Chem. Intermed. 26:253-267, 2000), and Cornelli et al. (Journal of Nutrition 131:3208-3211, 2001), which are incorporated herein by reference. This test is a spectrophotometric test that determines the concentration of hydroperoxides (ROOH). Such compounds are generated into the cells by the oxidative attack of reactive oxidative species (ROS) on a number of organic substrates (e. g. carbohydrates, lipids, amino acids, proteins, nucleotides etc.). During the test the hydroperoxides of a sample, e. g. the blood serum, after reacting with a chromogenic substrate develop a colored derivative (pink to red). Such colored complex is detectable and then quantifiable by a spectrophotometric methodic. Hydroperoxides concentration, which directly correlates with detected color intensity, is expressed as Carratelli Unit (CARR U), where 1 CARR U correspond to 0.08 mg/100 mL H₂O₂.

In the DROMs test, hydroperoxides of a sample are exposed to the same conditions of the Fenton's reaction to generate in vitro alkoxyl and peroxyl radicals. By diluting the sample with an acidic buffered solution (pH ˜4.8). At this conditions, iron previously bonded to serum proteins becomes available to catalyze the breakdown of blood hydroperoxides to alkoxyl and peroxyl radicals. A compound (chromogen) having the ability to change its color when oxidized by hydroperoxyl and alkoxyl radicals is then added to this solution. The chromogenic substrate used in the DROMs test is preferably N,N,-diethylparaphenylen-diamine, which is capable of being oxidized by hydroperoxyl and alkoxyl radicals, thus transforming itself in a pink to a red colored cation. The color development can be monitored spectrophotometrically at wavelength 505 or 546 nm. The concentration of colored complex is directly related to the hydroperoxides levels of the tested sample.

An automated DROM test is disclosed by Iamelle et al. (Clinical Chemistry and Laboratory Medicine 40(7):673-676, 2002). DROM tests are commercially available from Diacron International s.r.l. in Grosseto, Italy.

IL-6 concentration can be determined by various methods available in the prior art. Typically, an immunoassay, such as ELISA, is appropriate for determining IL-6 concentration. The availability of antibodies that are capable of specifically binding IL-6 has permitted the development of sensitive immunoassays of IL-6 concentration. Such antibodies can be obtained from Genzyme Corp. (Boston, Mass.), or from R&D Systems, Inc. (Minneapolis, Minn.).

Immunoassays are assay systems that exploit the ability of an antibody to specifically recognize and bind to a particular target molecule, which are used extensively in modern diagnostics (Fackrell, Clin. Immunoassay 8:213-219, 1985, which is incorporated herein by reference). A large number of different immunoassay formats have been described (Yolken, Rev. Infect. Dis. 4:35, 1982; Collins, In: Alternative Immunoassays, John Wiley & Sons, NY, 1985; Ngo et al., In: Enzyme Mediated Immunoassay, Plenum Press, NY, 1985, all of which are incorporated herein by reference).

Corcoran et al. (Clin. Chem. 37:1046, 1991), which is incorporated herein by reference, disclose an enzyme immunoassay for the quantification of IL-6 in serum. The assay is stated to be capable of detecting 2.6 pg/ml.

Other IL-6 immunoassay protocols have been described by Buyalos et al. (Feral. Steril. 57:1230-1234, 1992), and by Thavasu et al. (J. Immunol. Meth. 153:115-124, 1992), which are incorporated herein by reference. The assay of Buyalos et al. is used to measure IL-6 levels in follicular fluids with a detection limit of 50 pg/ml. The assay of Thavasu et al. is used to assay IL-6 in blood, and has a detection level of 70 pg/ml. A solid phase monoclonal immunoassay for IL-6 has also been described by Helle et al. (J. Immunol. Meth. 138:47-56, 1991), which is incorporated herein by reference.

Commercial immunoassay kits for IL-6 are also available (Human IL-6 ELISA kit, Cell Sciences, Inc., Canton, Mass.; IL-6 EIA and IL-6 ELISA kits, Cayman Chemicals, Ann Arbor, Mich.; Human High Sensitivity IL6 ELISA Kit, Abcam, Inc., Cambridge, Mass.; and Human IL-6 ELISA Ready-SET-Go!, eBioscience, Inc., San Diego, Calif.).

CRP concentration can be determined by various methods available in the prior art. Typically, an immunoassay, such as ELISA, is appropriate for determining CRP concentration. The availability of antibodies that are capable of specifically binding CRP has been developed to permit high sensitive immunoassays for CRP. Such antibodies and immunoassay kits can be obtained from Oxis International Inc. (Beverly Hills, Calif.), IBL America (Minneapolis, Minn.), or Phoenix Pharmaceuticals, Inc. (Burlingame, Calif.).

U.S. Pat. No. 6,838,250, which is incorporated herein by references, discloses a competitive immunoassay for CRP using a low affinity anti-CRP antibody and a antiidiotypic antibody raised against the low affinity anti-CRP antibody. Two ELISA formats are disclosed. A first format consists of the antiidiotypic antibody immobilized onto the plate surface and the HRP-labeled anti-CRP antibody competes with soluble CRP in a sample for sites on the immobilized antiidiotypic antibody. A second format uses the opposite orientation of reagents where the anti-CRP antibody is immobilized while the HRP-labeled antiidiotypic antibody and CRP in solution compete for antiCRP sites on the plate. Standard ELISA procedures were followed to immobilize antibody, block non-specific sites, titer labels, and for signal generation and detection. This patent also discloses methods for obtaining the low affinity anti-CRP antibody and the antiidiotypic antibody.

Other CRP immunoassay formats are disclosed in U.S. Pat. Nos. 4,902,630, 5,358,852; 5,272,258; and Shapiro et al. (Clin Chim Acta 180(3):285-92, 1989); Price et al. (Clin Chem Lab Med 37(2):109-13, 1999); Wood et al. (Clin Lab 2000;46(3-4):131-40); Roberts et al. (Clin Chem 46(4):461-8, 2000); Hutchinson et al. (Clin Chem 46(7):934-8, 2000), all of which are incorporated herein by reference.

U.S. Pat. No. 5,003,065, which is incorporated herein by reference, discloses an electrochemical method for measuring the concentration of CRP. This method is based on a membrane containing a protonated c-reactive protein specific binding compound complexed with a metal ion. When the membrane is exposed to the presence of CRP, the binding of CRP and the binding compound produces a change in the electrochemical potential across the membrane which is a direct function of the quantity of CRP bound by the binding compound. The patent also provides the protonated c-reactive protein specific binding compound to practice the electrochemical measuring method.

Various samples can be collected from a subject suspected of having ventricular arrhythmia risk. The sample can be whole blood, blood plasma, blood serum, or cell extract. The preferred samples are blood based, such as whole blood, blood plasma, and blood serum.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### Example 1

### Methods

To select patients at high risk for ventricular arrhythmia, this retrospective study was performed by examining patients either undergoing ICD implantation or generator exchange. This study protocol was approved by the Emory University Internal Review Board. These patients were enrolled in the Genetic Risk Assessment for Defibrillator Events (GRADE) trial, and were undergoing new ICD implant or who had undergone ICD placement or generator exchange within the last 5 years were enrolled from the four Emory University Hospitals. Patients met the inclusion criteria of being age 18 or older, able to give informed consent and had depressed left ventricular ejection fraction (LVEF) <30%. Exclusion criteria included patient refusal, patients with a life expectancy less than 6 months, patients who had ongoing class IV heart failure symptoms, patients who were post-cardiac transplant or with left ventricular assist devices. Demographic and medical information obtained on enrollment included: age, gender, race, history of smoking, medications, New York Heart Association (NYHA) class, etiology of heart disease, hypercholesterolemia, history of myocardial infarction (MI) history of coronary artery bypass (CABG) surgery, family history of heart disease, history of arrhythmias, history of syncope, echocardiogram results, cardiac catheterization results, nuclear imaging results, electrocardiograms, blood pressure, heart rate, electrolytes and date of ICD implantation surgery and any ICD generator exchanges.

Biomarker data: A single blood draw was performed at the time of enrollment and analyzed for markers of oxidative stress and inflammation in the Emory Biomarkers Core Laboratory. Markers used to measure oxidative stress were: ratios of oxidized to reduced glutathione (Eₕ GSH) and cystiene (Eₕ CySH) in plasma (thiol ratios)¹⁸ and derivatives of reactive oxygen species (DROMs)^{19;20;31}. Detailed methods to prevent rapid oxidation of samples have been delineated previously²¹. Blood was collected from an antecubital vein and transferred immediately to a micro-centrifuge tube containing 0.5 mL of a preservation solution of 100 mM serine-borate (pH 8.5) containing (per mL) 0.5 mg sodium heparin, 1 mg bathophenanthroline disulfonate sodium salt, and 2 mg iodoacetic acid. Use of this procedure minimizes auto-oxidation and hemolysis.²² All blood was drawn between 7:30 am and 3:00 pm in non-fasting patients. Following centrifugation to remove blood cells, aliquots (200 μL) were transferred to tubes containing 200 μL of 10% (w/v) perchloric acid containing 0.2 M of boric acid and 10 μM γ-Glu-Glu as internal standard. Samples were stored at −80° C. (<2 months) prior to further processing to form N-dansyl derivatives and analysis by HPLC with fluorescence detection. Reduced glutathione, cystine, and cystiene levels in plasma were greater than 1,000 times the level of detection (˜1 nM). Oxidized glutathione levels were approximately 10 times this limit. Previous data have shown stable measurements over this length of storage²³. Metabolites were identified by co-elution with standards, and quantificed by integration relative to the internal standard. Samples from control and AF patients were treated identically.

We calculate the redox states (Eₕ) of the thiol/disulfide pools were calculated with the Nernst equation, Eₕ=Eₒ+RT/nF ln[disulfide]/[thiol]². Eₒ is the standard potential for the redox couple, R is the Rydberg constant, T is the absolute temperature, n is 2 for the number of electrons transferred, and F is Faraday's constant. The standard potential Eₒ used for the glutathione and cystiene redox couples was −264 mV and −250 mV, respectively²⁴. Less negative Eₕ numbers imply a more oxidized state. DROMs were measured in Carr units with higher values indicating higher levels of oxidative stress. DROMs (Diacron International, Grosseto, Italy) and inflammatory markers, high sensitivity C-reactive protein (hsCRP; Life Diagnostics, West Chester, Pa.), interleukin-1-β (IL-1β; R&D Systems, Minneapolis, Minn.), interleukin-6 (IL-6; R&D Systems), and tumor necrosis factor α (TNFα; R&D Systems), were measured using commercially available kits.

Ventricular arrhythmias: Routine device interrogations and chart review were performed. All history of appropriate therapies for ventricular fibrillation (VF) or ventricular tachycardia (VT) were recorded. Dates, times, types and number of therapies were all documented. As the study was retrospective, there was no standardization of ICD programming; some patients had antitachycardia pacing (ATP) programmed on and some did not. Thus both ATP and shock therapies were recorded (further referred to as “ICD events”). All therapies were adjudicated by an independent cardiologist as appropriate therapy for ventricular arrhythmias or inappropriate therapy, for a non-VT/VF. Only appropriate therapies documented to be for ventricular arrhythmias were included in the analysis. Due to high variability of event rates and discrepancy in follow up time, events were analyzed as a function of time and analyzed as “event-months”.

Data analysis: Statistical analysis was performed using SPSS software version 14.0 (SPSS Inc., Chicago, Ill. 60606). Baseline characteristics of patients who received and did not receive ICD therapies were compared using a paired t-test for continuous variables (expressed as mean±SD) and Fisher's exact test for categorical variables. Baseline characteristics of patients who received and did not receive statins were compared using a paired t-test for continuous variables (expressed as mean±SD) and Fisher's exact test for categorical variables. Marker data were presented as the mean±SD, except as noted. All statistical tests were two-tailed, and significance was taken to be ρ≦0.05. Patient characteristics and all oxidative and inflammatory markers were examined for links to ICD events using Pearson's correlation coefficients. Multivariate models were used to examine the association between each oxidative marker and the occurrence of ICD therapies while controlling for other inflammatory markers and significant characteristics. Due to the wide range of follow up times, events were examined as a function of time, in “event-months.”

### Results

304 patients were enrolled and had blood tests performed and received 3 months or more of follow up (range: 3 months to 135 months, mean 29 months). Demographic data is presented in Table 1.

TABLE 1
Baseline demographics

| Age | 62 ± 12 | |
|---|---|---|
| Gender | 252 men | (83%) |
| CAD | 196 | (65%) |
| DM | 114 | (38%) |
| ICD therapies | 68 | (23%) |
| Average EF | 20% ± 7% | |
| Statins | 175 | (58%) |
| Smokers | 202 | (67%) |
| Afib | 87 | (29%) |
| ACE | 177 | (58%) |
| ARB | 71 | (23%) |
| PPAR | 28 | (9.2%) |

| Biomarker | Value |
|---|---|
| CRP | 5.7 ± 4.67 |
| IL-6 | 4.3 ± 3.2 |
| IL1β | 0.52 ± 0.37 |
| TNF-α | 4.4 ± 2.8 |
| DROM | 383 ± 95 |
| EhGSH | −126 ± 13 |
| EhCYS | −66 ± 9 |

There were 252 men (83%) and 52 women (17%). Average age was 63±11, EF 20%±7%, 114 (38%) had diabetes, 175 (58%) were on statins, 234 (80%) had no ICD therapies, 200 (67%) were smokers. 196 (65%) had coronary artery disease (CAD). Medication use examined included ACE-inhibitors (177/58%), ARBs (71 23%) and PPARs (28, 9.2%), all of which are known to affect oxidative stress. Mean biomarker values were high for all patients (Table 1). Table 2 shows compares patients using statins to those who were not using statins.

TABLE 2

| | | |
|---|---|---|
| Statin Use | No Statin Use | |
| (n = 175) | (n = 129) | ρ |

| | | | |
|---|---|---|---|
| Age | 59 ± 13 | 65 ± 9 | .00 |

| | | | | | |
|---|---|---|---|---|---|
| Gender | 146 | (83%) | 106 | (82%) | .46 |
| DM | 101 | (57%) | 40 | (31%) | .055 |
| Smokers | 127 | (72%) | 75 | (58%) | .01 |
| CAD | 138 | (78%) | 58 | (45%) | .00 |

| | | | |
|---|---|---|---|
| EF | 20% ± 7% | 19% ± 7% | |
| CRP | 5.2 ± 4.4 | 6.3 ± 5.0 | .05 |
| DROM | 373 ± 87 | 397 ± 102 | .03 |
| IL1-β | 0.52 ± 0.37 | 0.53 ± 0.36 | .90 |
| IL-6 | 4.3 ± 3.4 | 4.5 ± 3.0 | .88 |
| TNF-α | 4.5 ± 3.0 | 4.3 ± 2.6 | .64 |
| EhGSH | −126 ± 12 | −126 ± 13 | .82 |
| EhCYS | −66 ± 9 | −67 ± 9 | .93 |

| | | | | | |
|---|---|---|---|---|---|
| Afib | 37 | (30%) | 50 | (29%) | .87 |

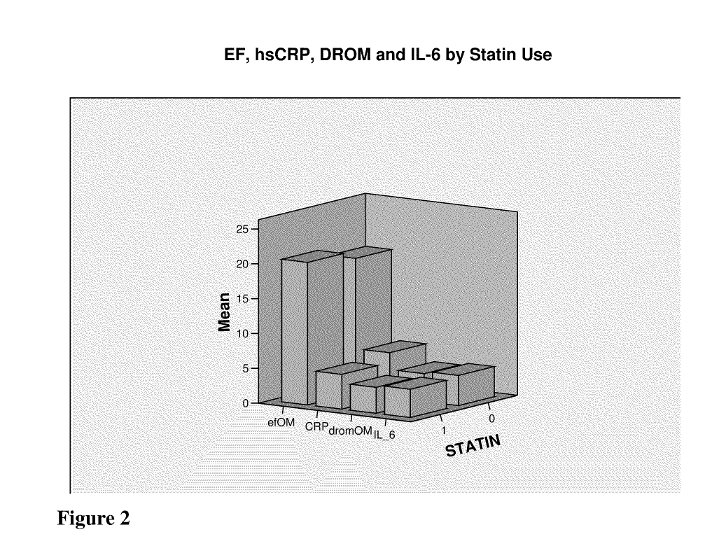

There is a significant difference in incidence of CAD (ρ=0.00) and cigarette smoking (ρ=0.01) in patients on statins. However, cigarette smoking correlates directly with CAD and is not an independent variable. DROM and hsCRP are significantly lower in the statin group. FIG. 2 shows EF, hsCRP, DROM and IL-6 by statin use. Table 3 shows the Pearson correlation coefficients and p values for the characteristics that correlated with ICD therapy events.

TABLE 3
ICD events*

| | Pearson's Correlation | |
|---|---|---|
| | Coefficient | ρ value |
| Age | −.058 | .37 |
| Gender | −.033 | .57 |
| DM | .052 | .37 |
| Cigs | 0.079 | .17 |
| CAD | 0.37 | .52 |
| EF | −.120 | .04 |
| CRP | .057 | .37 |
| DROM | .188 | .003 |
| IL-6 | .129 | .043 |
| IL1-β | −.065 | .30 |
| TNF-α | −.111 | .08 |
| EhGSH | .064 | .32 |
| EhCYS | −.005 | .94 |
| Statin | .037 | −.114 |

*Analysis by event-months

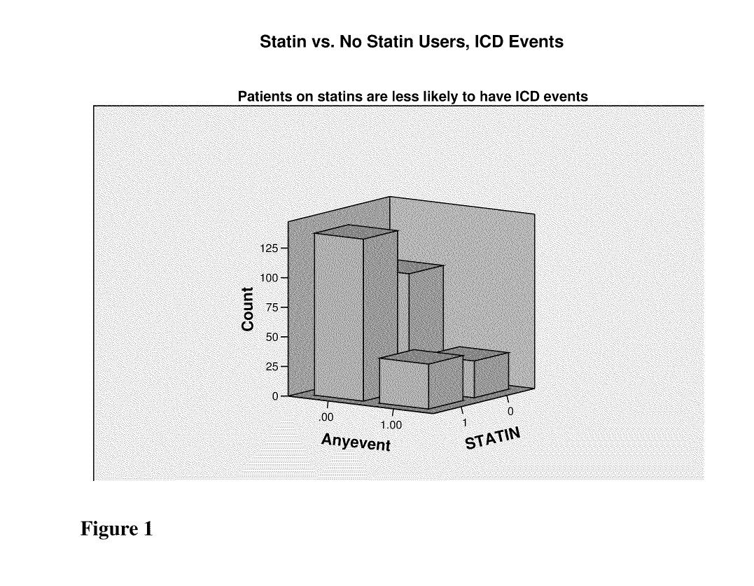

Ejection fraction, IL-6 levels, statins, TNF-α and DROMs all were significant. FIG. 1 shows the relationship between statin use and ICD events.

Multivariate cross-correlation analysis confirms the significant relationships of IL-6, DROMs, statins and EF with events. For IL-6: ρ=0.024, Pearson coefficient of 0.124; DROM: ρ=0.001, Pearson coefficient of 0.183; statins: ρ=0.047, Pearson coefficient of −0.107; TNF-α: ρ=0.040, Pearson coefficient of −0.112; and EF: ρ=0.015, Pearson coefficient of −0.132. Multivariate linear regression shows DROMs to be the dominant predictive factor of events, with a regression coefficient of 0.164 (ρ=0.026).

### Conclusions

For these high risk patients, we confirm the previous observation that statin medication use correlates with decreased rates of ventricular arrhythmias as measured by ICD therapies. We also demonstrate the independence of ejection fraction as a risk factor for ventricular arrhythmias. When we examined biomarkers to assess inflammation and oxidative stress burden, we found that hsCRP and DROM were decreased in the statin users group and that IL-6 and DROMS correlate with decreased event risk. IL-6 correlates with events, but not with statin use, suggesting IL-6 is unaffected by statin use. The only factor dependent on statin use and associated with decreased ICD events is DROM. That DROMs are the single most predictive indicator of future events, coupled with their statin correlation, provides strong evidence that the mechanism by which statins lower rates of ventricular arrhythmias is via their antioxidant effect.

### Discussion

Each patient considered had cardiac disease that qualified them for an ICD, giving them a high risk for ventricular arrhythmias. Our patient demographics do not differ significantly from those in the two large trials, previously cited, that demonstrate decreased ICD events with statins. Average patient age, gender, EF, rates of diabetes, and rates of ACE/ARB use were all similar. Perhaps unsurprisingly, a difference was seen in the rates of cigarette smokers. In the ischemic cardiomyopathy group (MADITII), the rate of smoking was 81%, in the non-ischemic group (DEFINITE), 38% were smokers. In our mixed ischemic and non-ischemic population, 67% of patients were smokers. Of our smokers, 76% had CAD, (and 73% of our CAD patients were smokers).

Our patients' biomarkers are elevated. Elevated inflammatory markers and markers of oxidative stress have been correlated with increased mortality in cardiac disease. hsCRP, for example, is considered a “high risk” marker (per AHA/CDC consensus document)²⁵ when the levels are >3.0 mg/dl. Our mean value was 5.7 In a study recently accepted for publication, we compare case-matched biomarkers for patients with and without atrial fibrillation. In that study we demonstrate that patients with AF are more oxidized compared to the controls. These ICD patients are similarly oxidized when compared our AF patients: DROMS are similar at 388 vs. 383, EhC −66 vs. −68, EhG −126 vs. −133. For our inflammatory markers hsCRP is higher (5.7 vs 3.9), as is IL-6 (5.5 vs. 4.3), TNF alpha is lower 4.4 vs. 6.4, and ILB was the same 0.5 vs. 0.5.

In these high-risk patients, statin use correlates with decreased arrhythmia risk. Decreases in DROMs correlate with decreased ICD events, and with statin use. This suggests that statin use decreases ventricular arrhythmias in part due to its anti-oxidant properties, possibly via ion channel alterations.

That IL-6 with does not change with statin use has been somewhat controversial in the literature. It has been previously documented to be unchanged with pravastatin, simvastatin, and atorvastatin in several studies²⁶⁻²⁸. Others, however, have seen a change in Il-6 with statin use.²⁹ As IL-6 is known to exhibit great circadian variation, this particular marker may be more sensitive to the variable follow-up time courses in our study. However, the lack of correlation with statin use further suggests that statins are acting through an IL-6 independent mechanism.

Measuring oxidative stress in humans is difficult because free radicals are reactive and thus short-lived. Products of free radical damage to DNA proteins and lipids may provide such markers. Additionally, measurements of O₂— generating enzymes can be easily quantified(already said measured). We chose several markers to examine: quantifiying thoi-disulfide redox couples, reduced and oxidized glutathione disulfide, and cysteine/cystine ratios. These redox states represent plasma oxidation state. To reflect the lipid compartment, we used a measure of plasma lipid peroxides known as the d-ROMs test. The positive correlation of reduced ICD events with DROMs may reflect changes in the lipid compartment, as opposed to the other markers of oxidative stress, which reflect changes in plasma oxidative stress. This finding demonstrates that the tissue oxidative state and the plasma oxidative state are not necessarily equivalent. That DROMs reflect the tissue state, and are significant is further circumstantial evidence to support a tissue-level mechanistic change.

### Example 2

### Methods

We evaluated 243 patients with systolic heart failure (left ventricular ejection fraction (LVEF) ≦30%) and implantable cardioverter-defibrillators (ICDs) enrolled in the Genetic Risk Assessment of Defibrillator Events study. Demographic data, biomarker levels, and appropriate ICD shocks and anti-tachycardia pacing (ATP) for VT/VF were compared between African American (AA) and Caucasian patients. Table 4 shows the clinical characteristics of the patients. Blood was analyzed for derivatives of reactive oxygen metabolites (DROM), high sensitivity C-reactive protein (CRP), and interleukin-6 (IL-6).

TABLE 4

| | African American | | Caucasian | |
|---|---|---|---|---|
| | 25.1% (61) | | 74.9% (182) | |
| Male gender | 72.1% | (44) | 85.2% | (155) |
| NICM | 39.3% | (24) | 18.7% | (34) p < 0.001 |
| Atrial fibrillation | 34.4% | (21) | 39.0% | (71) |
| Diabetes | 36.1% | (22) | 34.6% | (63) |
| Age (years) | 57.3 ± 12.8 | | 64.2 ± 10.8 p = 0.01 | |
| LVEF (%) | 19.8 ± 7.5 | | 21.1 ± 6.2 | |
| ACC/AHA HF Class | 2.11 ± 0.8 | | 2.05 ± 0.7 | |
| Creatinine (mg/dl) | 1.3 ± 0.4 | | 1.4 ± 1.0 | |
| Weight (lbs.) | 197.8 ± 45.1 | | 198.6 ± 42.7 | |
| Previous smoking | 52.5% | (32) | 74.7% | (136) p = 0.002 |
| Currently smoking | 3.3% | (2) | 13.2% | (24) p = <0.0001 |

| | | |
|---|---|---|
| Pack-years smoking | 29.8 ± 25.4 | 43.2 ± 31.7 p = 0.03 |

### Results

Subjects were 74.9% (182) Caucasian, 25.1% (61) AA, and median follow-up was 28.4 months. Compared to Caucasians, AA were more likely to have NICM (39.3% vs. 18.7%; p<0.001) and were younger (57 vs. 64 years; p=0.01). There was no significant difference in mean LVEF (18% vs. 20%; p=0.054), ACC/AHA heart failure class (2.11 vs. 2.07, p=0.69), incidence of diabetes, or chronic kidney disease.

Table 5 shows inflammatory and oxdidative stress markers in the patients being studied. AA had higher mean levels of CRP (8.4±5.7 vs. 5.1±4.6 ng/dl; p=0.01), IL-6 (5.8±4.1 vs. 4.2±4.0 ng/dl; p=0.005) and DROM (440±131 vs. 357±84 Carr; p=0.001). Differences in ICD therapy rates, cardiac hospitalizations, and mortality were independent of ischemic versus nonischemic cardiomyopathy. Of the 61 AA patients in this study, 31 were matched to Caucasians for factors known to influence oxidative stress (age, diabetes, and smoking) and heart failure status (LVEF, heart failure class, and heart failure etiology). In these matched patients AA had higher mean levels of CRP (7.6±6.0 vs. 4.7±4.2 ng/dl; p=0.031) and IL-6 (5.6±3.7 vs. 3.4±2.2 ng/dl; p=0.006) but no significant difference in DROM.

TABLE 5

| | African American | Caucasian | |
|---|---|---|---|
| | 25.1% (61) | 74.9% (182) | p value |
| CRP (ng/dl) | 8.4 ± 5.7 | 5.1 ± 4.6 | 0.01 |
| IL-6 (ng/dl) | 5.8 ± 4.1 | 4.2 ± 4.0 | 0.005 |
| TNF-alpha | 4.29 ± 2.9 | 4.84 ± 5.0 | 0.35 |
| IL1-beta | 0.54 ± 0.45 | 0.57 ± 0.50 | 0.98 |
| DROM (Carr) | 440 ± 131 | 357 ± 84 | 0.001 |
| EhGSH | −121.0 ± 12.1 | −126.1 ± 12.3 | 0.90 |
| EhCyS | −67.8 ± 7.9 | −67.9 ± 8.6 | 0.54 |

### Example 3

### Methods

Demographic data, biomarkers, and appropriate ICD shocks and anti-tachycardia pacing (ATP) were examined in 232 patients (LVEF ≦30%) with ICDs enrolled in the Genetic Risk Assessment of Defibrillator Events study. Table 6 shows the clinical and demographic data for the patients. Blood was analyzed for derivatives of reactive oxygen metabolites (DROM), high sensitivity C-reactive protein (CRP), and interleukin-6 (IL-6).

TABLE 6

| Women | Men | |
|---|---|---|
| 19.8% (46) | 80.2% (186) | p value |

| | | | |
|---|---|---|---|
| Age (years) | 57.7 ± 10.3 | 62.8 ± 12.1 | 0.009 |

| | | | | | |
|---|---|---|---|---|---|
| Caucasian | 54.3% | (25) | 76.9% | (143) | 0.003 |
| Hypertension | 71.7% | (33) | 66.1% | (123) | 0.599 |
| Ischemic | 37.0% | (17) | 67.2% | (125) | <0.0001 |
| cardiomyopathy | | | | | |

| | | | |
|---|---|---|---|
| LVEF (%) | 19.6 ± 6.9 | 20.0 ± 7.1 | 0.74 |

| | | | | | |
|---|---|---|---|---|---|
| Diabetes | 41.3% | (19) | 33.9% | (63) | 0.39 |

| | | | |
|---|---|---|---|
| ACC/AHA heart | 1.9 ± 0.7 | 2.1 ± 0.7 | 0.09 |
| failure class | | | |

| | | | | | |
|---|---|---|---|---|---|
| Smoking history | 39.1% | (18) | 69.9% | (130) | <0.0001 |

### Results

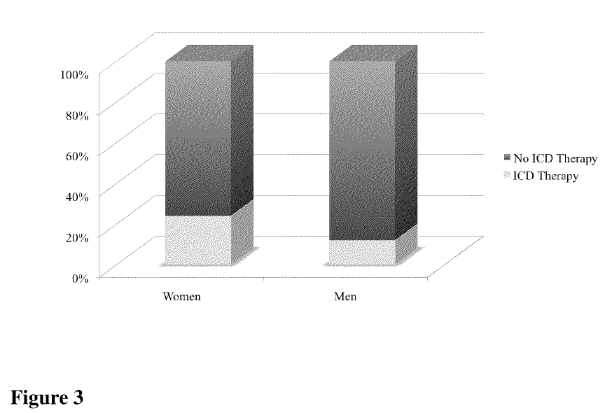

Subjects were 20% (46) women and 80% (186) men. Mean follow-up time was 17 (range 3-48) months. LVEF did not significantly differ (19%±7% vs. 20%±7%, p=0.74) nor did incidence of diabetes, hypertension, or heart failure classification. (Table 6) Women were younger than men (58±10 vs. 62±12 years, p<0.001) and had lower incidence of ischemic cardiomyopathy (37% [17] vs. 67% [125], p<0.001). Women had higher levels of DROM (452±102 vs. 365±95 Carr, p<0.001) and CRP (7.7±5.3 vs. 5.6±5.0 ng/dl, p=0.015). (Table 7) IL-6 levels did not significantly differ. Appropriate ICD shocks occurred more frequently in women (24% [11] vs. 12% [23], p=0.015) (FIG. 3), while event rates of appropriate ICD therapies over time were significantly higher in women (0.84 vs. 0.21 events/year, p=0.011). Of the 46 women in this study, 32 were matched to men for clinical factors known to influence oxidative stress (age, diabetes, hypertension, and cigarette smoking). In these matched patients, as shown in Table 7, women had higher levels of DROM (452±86 vs. 401±83 Carr, p=0.007), but no significant differences in IL-6 or CRP.

TABLE 6

| | Women | Men | |
|---|---|---|---|
| | 19.8% (46) | 80.2% (186) | p value |
| CRP (ng/dl) | 7.7 ± 5.3 | 5.6 ± 5.0 | 0.015 |
| DROM (Carr) | 452 ± 102 | 365 ± 95 | <0.001 |
| IL-6 (ng/dl) | 4.6 ± 4.0 | 4.7 ± 4.1 | 0.895 |

TABLE 7

| | Women | Men | |
|---|---|---|---|
| | 50% (32) | 50 (32) | p value |
| CRP (ng/dl) | 452 ± 86 | 399 ± 85 | 0.017 |
| DROM (Carr) | 8.2 ± 5.3 | 7.0 ± 5.3 | 0.38 |
| IL-6 (ng/dl) | 4.8 ± 4.2 | 5.4 ± 3.5 | 0.50 |

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

### REFERENCE LIST

(These references are incorporated herein by reference):
1. State Specific mortality from sudden cardiac death—United States 1999. Centers for Disease Control and Prevention. 123-6. 2-15-2002. MMRW.

### Ref Type: Report

2. Zheng Z J, Croft J B, Giles W H, Mensah G A. Sudden cardiac death in the United States, 1989 to 1998. Circulation. 2001; 104:2158-2163.
3. Bayes d L, Coumel P, Leclercq J F. Ambulatory sudden cardiac death: mechanisms of production of fatal arrhythmia on the basis of data from 157 cases. Am Heart J. 1989; 117:151-159.
4. Vyas A K, Guo H, Moss A J, Olshansky B, McNitt S A, Hall W J, Zareba W, Steinberg J S, Fischer A, Ruskin J, Andrews M L. Reduction in ventricular tachyarrhythmias with statins in the Multicenter Automatic Defibrillator Implantation Trial (MADIT)-II. J Am Coll Cardiol. 2006; 47:769-773.
5. Goldberger J J, Subacius H, Schaechter A, Howard A, Berger R, Shalaby A, Levine J, Kadish A H. Effects of statin therapy on arrhythmic events and survival in patients with nonischemic dilated cardiomyopathy. J Am Coll Cardiol. 2006; 48:1228-1233.
6. Stoll L L, McCormick M L, Denning G M, Weintraub N L. Antioxidant effects of statins. Drugs Today (Barc). 2004; 40:975-990.
7. Delbosc S, Morena M, Djouad F, Ledoucen C, Descomps B, Cristol J P. Statins, 3-hydroxy-3-methylglutaryl coenzyme A reductase inhibitors, are able to reduce superoxide anion production by NADPH oxidase in THP-1-derived monocytes. J Cardiovasc Pharmacol. 2002; 40:611-617.
8. Choi M, Rolle S, Rane M, Haller H, Luft F C, Kettritz R. Extracellular signal-regulated kinase inhibition by statins inhibits neutrophil activation by ANCA. Kidney Int. 2003; 63:96-106.
9. Dechend R, Fiebeler A, Park J K, Muller D N, Theuer J, Mervaala E, Bieringer M, Gulba D, Dietz R, Luft F C, Haller H. Amelioration of angiotensin II-induced cardiac injury by a 3-hydroxy-3-methylglutaryl coenzyme a reductase inhibitor. Circulation. 2001; 104:576-581.
10. Amin-Hanjani S, Stagliano N E, Yamada M, Huang P L, Liao J K, Moskowitz M A. Mevastatin, an HMG-CoA reductase inhibitor, reduces stroke damage and upregulates endothelial nitric oxide synthase in mice. Stroke. 2001; 32:980-986.
11. Laufs U, Fata V L, Liao J K. Inhibition of 3-hydroxy-3-methylglutaryl (HMG)-CoA reductase blocks hypoxia-mediated down-regulation of endothelial nitric oxide synthase. J Biol Chem. 1997; 272:31725-31729.
12. Stoll L L, McCormick M L, Denning G M, Weintraub N L. Antioxidant effects of statins. Timely Top Med Cardiovasc Dis. 2005; 9:E1.
13. Korantzopoulos P, Kolettis T M, Kountouris E, Dimitroula V, Karanikis P, Pappa E, Siogas K, Goudevenos J A. Oral vitamin C administration reduces early recurrence rates after electrical cardioversion of persistent atrial fibrillation and attenuates associated inflammation. Int J Cardiol. 2005; 102:321-326.
14. Korantzopoulos P, Kolettis T M, Galaris D, Goudevenos J A. The role of oxidative stress in the pathogenesis and perpetuation of atrial fibrillation. Int J Cardiol. 2007; 115:135-143.
15. Kim Y H, Lim D S, Lee J H, Shim W J, Ro Y M, Park G H, Becker K G, Cho-Chung Y S, Kim M K. Gene expression profiling of oxidative stress on atrial fibrillation in humans. Exp Mol Med. 2003; 35:336-349.
16. Fukuda K, Davies S S, Nakajima T, Ong B H, Kupershmidt S, Fessel J, Amarnath V, Anderson M E, Boyden P A, Viswanathan P C, Roberts L J, Balser J R. Oxidative mediated lipid peroxidation recapitulates proarrhythmic effects on cardiac sodium channels. Circ Res. 2005; 97:1262-1269.
17. Ward C A, Giles W R. Ionic mechanism of the effects of hydrogen peroxide in rat ventricular myocytes. J Physiol. 1997; 500 (Pt 3):631-642.
18. Jones D P, Mody V C, Jr., Carlson J L, Lynn M J, Sternberg P, Jr. Redox analysis of human plasma allows separation of pro-oxidant events of aging from decline in antioxidant defenses. Free Radic Biol Med. 2002; 33:1290-1300.
19. Cesarone M R, Belcaro G, Carratelli M, Comelli U, De Sanctis M T, Incandela L, Barsotti A, Terranova R, Nicolaides A. A simple test to monitor oxidative stress. Int Angiol. 1999; 18:127-130.
20. Abramson J L, Hooper W C, Jones D P, Ashfaq S, Rhodes S D, Weintraub W S, Harrison D G, Quyyumi A A, Vaccarino V. Association between novel oxidative stress markers and C-reactive protein among adults without clinical coronary heart disease. Atherosclerosis. 2005; 178:115-121.
21. Jones D P, Carlson J L, Samiec P S, Sternberg P, Jr., Mody V C, Jr., Reed R L, Brown L A. Glutathione measurement in human plasma. Evaluation of sample collection, storage and derivatization conditions for analysis of dansyl derivatives by HPLC. Clin Chim Acta. 1998; 275:175-184.
22. Jones D P, Carlson J L, Samiec P S, Sternberg P, Jr., Mody V C, Jr., Reed R L, Brown L A. Glutathione measurement in human plasma. Evaluation of sample collection, storage and derivatization conditions for analysis of dansyl derivatives by HPLC. Clin Chim Acta. 1998; 275:175-184.
23. Jones D P, Carlson J L, Samiec P S, Sternberg P, Jr., Mody V C, Jr., Reed R L, Brown L A. Glutathione measurement in human plasma. Evaluation of sample collection, storage and derivatization conditions for analysis of dansyl derivatives by HPLC. Clin Chim Acta. 1998; 275:175-184.
24. Jones D P, Carlson J L, Samiec P S, Sternberg P, Jr., Mody V C, Jr., Reed R L, Brown L A. Glutathione measurement in human plasma. Evaluation of sample collection, storage and derivatization conditions for analysis of dansyl derivatives by HPLC. Clin Chim Acta. 1998; 275:175-184.
25. Fortmann S P, Ford E, Criqui M H, Folsom A R, Harris T B, Hong Y, Pearson T A, Siscovick D, Vinicor F, Wilson P F. CDC/AHA Workshop on Markers of Inflammation and Cardiovascular Disease: Application to Clinical and Public Health Practice: report from the population science discussion group. Circulation. 2004; 110:e554-e559.
26. Jialal I, Stein D, Balis D, Grundy S M, Adams-Huet B, Devaraj S. Effect of hydroxymethyl glutaryl coenzyme a reductase inhibitor therapy on high sensitive C-reactive protein levels. Circulation. 2001; 103:1933-1935.
27. Lindholm M W, Nilsson J. Simvastatin stimulates macrophage interleukin-1beta secretion through an isoprenylation-dependent mechanism. Vascul Pharmacol. 2007; 46:91-96.
28. Erikstrup C, Ullum H, Pedersen B K. Short-term simvastatin treatment has no effect on plasma cytokine response in a human in vivo model of low-grade inflammation. Clin Exp Immunol. 2006; 144:94-100.
29. Yokota K, Miyazaki T, Hirano M, Akiyama Y, Mimura T. Simvastatin inhibits production of interleukin 6 (IL-6) and IL-8 and cell proliferation induced by tumor necrosis factor-alpha in fibroblast-like synoviocytes from patients with rheumatoid arthritis. J Rheumatol. 2006; 33:463-471.
31. Cornelli U, Terranova R, Luca S, Cornelli M, Alberti A. Bioavailability and antioxidant activity of some food supplements in men and women using the D-Roms test as a marker of oxidative stress. J Nutrition 2001; 131:3208-3211.

What is claimed is:

1. A method for assessing or diagnosing a risk of ventricular arrhythmia in an individual, wherein the individual is an African American or a woman, the method comprising the step of
a. obtaining a sample from a patient;
b. determining DROMs and/or IL-6 and/or CRP concentration in the sample; and
c. assessing or diagnosing the risk of ventricular arrhythmia from said concentration

2. The method of claim 1, wherein the concentration of IL-6 or CRP is determined by immunoassay.

3. The method of claim 1, wherein the concentration of DROM is determined by measuring the amount of hydroperoxides in the blood.

4. The method of claim 1, wherein an increased risk of heart disease is assessed or diagnosed when the concentration of DROM and/or IL-6 and/or CRP is elevated when compared to normal individuals.

5. The method of claim 1, wherein an increased risk of heart disease is assessed or diagnosed when the concentration of DROM and/or IL-6 and/or CRP is increased or is abnormally high.

6. The method of claim 1, wherein the sample is blood.

7. The method of claim 6, where in the sample is whole blood, blood serum, or blood plasma.

8. A method for monitoring the treatment of an individual with ventricular arrhythmia risk, wherein the individual is an African American or a woman, the method comprising the steps of
administering a pharmaceutical composition for treating heart disease to the individual; and
determining the blood level of DROM and/or IL-6 and/or CRP in the individual.

9. The method of claim 8, wherein a decrease in DROM and/or IL-6 and/or CRP levels indicate effectiveness of the pharmaceutical composition.

10. The method of claim 8, wherein the levels of IL-6 and/or CRP is determined by immunoassay.

11. The method of claim 8, wherein the levels of DROM is determined by measuring the amount of hydroperoxides in the blood.

12. The method of claim 8, wherein the sample is blood.

13. The method of claim 12, where in the sample is whole blood, blood serum, or blood plasma.

14. A method for screening for an agent capable of decreasing the risk of ventricular arrhythmia, wherein the individual is an African American or a woman, the method comprising the steps of
exposing an individual to the agent; and
determining the blood level of DROM and/or IL-6 and/or CRP in the individual.

15. The method of claim 14, wherein a decrease in DROM and/or IL-6 and/or CRP levels indicate effectiveness of the agent in decreasing the risk of ventricular arrhythmia.

16. The method of claim 14, wherein the levels of IL-6 and/or CRP is determined by immunoassay.

17. The method of claim 14, wherein the levels of DROM is determined by measuring the amount of hydroperoxides in the blood.

18. The method of claim 14, wherein the sample is blood.

19. The method of claim 18, where in the sample is whole blood, blood serum, or blood plasma.
